(19) United States
(12) Patent Application Publication
(10) Pub. No.: US 2002/0077579 A1
(43) Pub. Date: Jun. 20, 2002

(54) Sterile bicarbonate-free dialysis concentrate solutions

(76) Inventor: Sheldon Tobe, Toronto (CA)

Correspondence Address: IVOR M HUGHES, 175 COMMERCE VALLEY DRIVE WEST, SUITE 200, THORNHILL, ON, L3T7P6, CA

(21) Appl. No.: 10/020,881

(22) Filed: Dec. 19, 2001

Related U.S. Application Data
(60) Provisional application No. 60/256,492, filed on Dec. 20, 2000.

(51) Int. Cl. A61M 37/00; C02F 1/44; B01D 11/00

(52) U.S. Cl. 604/5.01; 210/650; 210/646

(57) ABSTRACT

The present invention relates to a sterile calcium-free bicarbonate-free dialysis concentrate composition for use in conjunction with regional citrate anti-coagulation in the context of either dialysis or hemofiltration. Methods of use of the sterile calcium free bicarbonate-free dialysis concentrate compositions are also disclosed. The bicarbonate-free concentrate takes into account the fact that citrate is hepatically metabolized into bicarbonate, thus minimizing metabolic complications.

### FIELD OF THE INVENTION

The present invention relates to a sterile concentrate dialysis solution. More particularly, it relates to a sterile calcium-free bicarbonate-free concentrate solution for use in dialysis and hemofiltration.

### BACKGROUND OF THE INVENTION

The purification of blood and separation of fluids using dialysis can be advantageously used in many medical applications, particularly conditions where renal function has significantly declined. Dialysis removes wastes from blood through a semipermeable membrane by diffusive or convective processes. There are two principal dialysis methods used to support patients requiring renal replacement therapy: hemodialysis and peritoneal dialysis Hemodialysis, involves the removal of solutes and fluids (such as urea, creatinine and uric acid) from the blood through a dialysis membrane by diffusion into a dialysate. The dialysis membrane is a semipermeable membrane which is typically made of cellulose. Blood solutes containing the waste permeate through the membrane and into a dialysis solution or dialysate formulated to control solute net movement through the membrane.

In the chronic hemodialysis setting, processes which have been developed and are commonly used provide bicarbonate dialysis using a highly sophisticated machine which can be monitored by a team. Dialysis provided in the intensive care setting for patients with an acute loss of kidney function has traditionally been provided with a chronic hemodialysis machine, brought into the unit and operated by one dialysis nurse per patient, in addition to the patient's intensive care nurse.

Hemodialysis can be either continuous or intermittent. Intermittent hemodialysis involves short intensive periods of treatment on alternate days, while continuous hemodialysis involves continuous fluid removal and continuous blood purification, often with a machine dedicated for this purpose.

Due to resource limitations dialysis often must be condensed into a period of hours and may be limited to less than daily treatments leading to large fluctuations in levels of the substances removed from the patient. These fluctuations may adversely affect patient outcomes. A dialysis therapy which comes closest to normal kidney function, by operating continuously may improve patient outcomes and shorten intensive care stays. This has led to the adoption of continuous modalities of renal replacement therapy (CRRT) in the intensive care setting.

Continuous renal replacement therapy (CRRT) is dialysis continued 24 hours a day. Unlike chronic hemodialysis there are no standardized equipment or processes for CRRT. To simplify the equipment necessary, CRRT does not use dialysate from concentrate, but uses pre-made dialysate, usually peritoneal dialysis solution. This solution is sterile and is buffered by lactate. The dialysis solution to which blood is exposed through this membrane should have the same electrolyte composition of normal serum or it may induce fatal electrolyte abnormalities. Its use with dialysis filters requires at a minimum the absence of pyrogens. If the solution is to be given intraperitoneally or intravenously it must be sterile and pyrogen free.

The electrolyte composition of all dialysis solutions may vary but in a narrow range. The major cationic electrolyte component is sodium, usually at the concentration it is found in serum 140 (mmol/L, mEq/L). Other cations include calcium (2.5 mmol/L, 5.0 mEq/L) and magnesium (0.75 mmol/L, 1.5 mEq/L). The major anion is chloride whose concentration is determined by the net of the cationic charge constituents less the anionic buffer. The dialysis solutions used in all forms of dialysis contain buffers in an attempt to correct metabolic acidosis. Common buffers used include bicarbonate, lactate and acetate buffers.

Bicarbonate buffer is a preferred buffer for dialysis since bicarbonate is the physiological buffer of the body. However, pre-made mixtures of bicarbonate buffered solutions are difficult to sterilize and store because released carbonate will precipitate with calcium if present. Attempts have been made to stabilize calcium, for example with glycylglycine (U.S. Pat. No. 5,211,643 to Reinhardt et al). Continuous dialysis against an agent such as glycylglycine produces levels in the blood close to those present in the dialysate. The effect of long term exposure to stabilizing agents such as glycylglycine is unknown (Yatzidis et al. Nephron., 64:27-31, 1993).

Furthermore, sugars in a dialysis solution will caramelize during heat sterilization and prolonged exposure if kept at neutral or higher pH (7.4). Therefore sugar containing dialysis solution is kept at low pH. For example, pH 5.4 for most peritoneal dialysis solutions. The low pH is believed to be the source of pain patients suffer after instillation of a fresh bag of peritoneal dialysis solution. Low pH solutions are known to reduce the effectiveness of peritoneal immunologic defences. The safety of using low pH solutions for dialysis or hemofiltration during CRRT has not been studied.

Also, during preparation and storage of a bicarbonate buffered solution, CO2 is released from the solution, changing the bicarbonate concentration and pH of the solution. It is therefore necessary for bicarbonate containing solutions to be stored in glass or CO2 impermeable plastic containers. The following solutions have been proposed to control the CO2 content of the bicarbonate solution for peritoneal dialysis: storage in a powder form until use; use of an impermeable barrier between calcium containing and bicarbonate containing portions; and addition of buffers such as histidine or glycylglycine (H. Yatzidis, Nephron 64:27-31, 1993).

Dialysis care has become process driven to maximize the quality of the dialysis and to minimize costs. Hemodialysis machines have been developed which can prepare dialysis solution online from a single concentrate and clean water provided from a central reverse osmosis system. To get around the stability problems associated with calcium and bicarbonate, acetate was substituted for bicarbonate. Acetate hemodialysis was carried out until evidence showed the deleterious effects of acetate on dialysis patients, particularly with the use of the newer more biocompatible dialysis membranes (F. H. Leenen, Artificial Organs 8:411-417, November 1994).

Dual proportioning dialysis machines have been developed and employed at great expense to provide bicarbonate dialysis. These machines solve the calcium bicarbonate instability problem by keeping the bicarbonate and acid concentrates separate until the time of dialysis. Although micro precipitation may occur immediately after mixture, clinically this is not a concern even over a 72 hour period (Leblanc et al, 1995). However, because of this precipitation bicarbonate dialysis machines must have acid rinses on a regular basis.

Separate batches of concentrates have been used using split bags which contain calcium and magnesium on the one hand, and the bicarbonate on the other hand to prevent precipitation (U.S. Pat. No. 4,630,727 to Feriani et al).

A method was been developed to allow an older single proportioning chronic dialysis machine to produce bicarbonate dialysis from concentrate using calcium free bicarbonate concentrate adding the calcium back into the blood by an infusion pump. This method for chronic dialysis was reported by Kaye et al, but was not adopted outside of Kaye's unit in Montreal. (M. Kaye et al., Clinical Nephrology 31:132-138, 1989; M. Kaye and D. Fisher, Clinical Nephrology 34:84-87, 1990; and M. Kaye, Clinical Nephrology 40:221-224, 1993). Calcium is infused distal to the dialyzer into the drip chamber using an infusion pump and is a component of the dialysate. In Kaye's studies, the patient's are not critically ill and his system is set up for chronic hemodialysis, not for acute hemodialysis. The concentrate used by Kaye is not sterile. Furthermore, Kaye's system is used for intermittent, but not for continuous dialysis.

Acute renal failure in critically ill patients, which is generally accompanied by metabolic derangements and high overall mortality, poses significant challenges for renal replacement therapy. Acute intermittent hemodialysis has been the conventional therapy. Bicarbonate dialysate which is typically used in acute intermittent hemodialysis is not sterile but only clean.

Problems with the rapid removal of fluid and changes in electrolytes which occur during high efficiency short term intermittent hemodialysis have led to the development and use of continuous renal replacement therapies (CRRT) for critically ill patients (P. Y. W. Tam et al., Clinical Nephrology 30:79-85, 1988 and E. F. H. Van Bommel et al, Am. J. Nephrol. 15:192-200, 1995). Solute and volume removal are slow and continuous during CRRT eliminating the large shifts occurring between body compartments during intermittent hemodialysis, which may lead to hypotension and interfere with renal recovery (E. F. H. Van Bommel, Nephrol. Dial. Transplant. 1995 Editorial Comments, p. 311). CRRT techniques include peritoneal dialysis, continuous arterio-venous and veno-venous ultrafiltration, hemofiltration, hemodialysis and hemodiafiltration. Traditionally CRRT has used peritoneal dialysis solution as the dialysate and infusate.

Lactate containing peritoneal dialysis solution has been used in CRRT dialysate with some success (Baxter and Gambro solutions). Lactate is stable with calcium and is stable at low pH (5.4). Lactate is metabolised by the intact functioning liver into bicarbonate, the body's natural buffer. However, lactate infusions are known to induce panic in susceptible individuals and may alter metabolism to favour catabolism over anabolism (R. L. Veech et al.). Its safety in CRRT dialysis has not been tested. However, its use as a buffer in peritoneal dialysis solution is universal and appears to be tolerated, except for abdominal pain and possible immunologic effects; there is mounting evidence that exposure to large amounts of lactate, particularly in the racemic form, may not be benign. Lactate included in these solutions is of the racemic form.

In intensive care patients, such as patients who have developed hypotension and lactic acidosis, lactate from the dialysis solution may not be metabolized to bicarbonate because of liver dysfunction, and when the dialysate lacks bicarbonate, acidosis may be worsened due to bicarbonate removal during dialysis. (A. Davenport et al., Nephron 1991:59:461-465, 1991 and M. Leblanc et al., Am. J. Kid. Dis. 26:910-917, 1995). For acute hemodialysis in the intensive care unit CRRT typically uses lactate based sterile solutions as dialysate and infusate (peritoneal dialysis solution). Research into methods to provide bicarbonate dialysate have been ongoing. Recently, a method was reported for providing non-sterile calcium bicarbonate dialysate for patients in the intensive care undergoing CRRT (M. Leblanc, AJKD 26(6):910-917, 1995). Non-sterile bicarbonate dialysis solutions can be produced in the chronic hemodialysis unit or hospital pharmacy and carried to the intensive care unit. These methods are labour intensive, unregulated, non sterile, not pyrogen free, expensive and may lack sufficient quality control. Unlike chronic hemo- or peritoneal dialysis, which are process driven and carried out in a uniform, cost effective quality controlled manner, CRRT is carried out in many different modalities specific to each intensive care unit.

It is important to use a sterile dialysis solution in CRRT in order to avoid pyrogenic reactions caused by bacteria and endotoxin contamination of the dialysate solution. It is also important to have a solution which is readily available for use. While sterile lactate or acetate based dialysis solutions may be used in CRRT they suffer from the disadvantages discussed above. It has been suggested that bicarbonate dialysate may be preferable to lactate or acetate-based solutions (M. Leblanc et al., Am. J. Kid. Dis. 26:910-917, 1995). However, it has not been possible to provide a sterile and readily available bicarbonate solution for CRRT due to the problems discussed above with bicarbonate solutions.

Furthermore, CRRT requires the addition of an anti-coagulent to the dialysate to prevent thrombosis. Standard techniques use systematic heparin as an anti-coagulent. However, may critically ill patients cannot tolerate heparin due to hemorrhage, severe coagulopathy, or heparin induced thrombocytopenia. Recently, methods for regional anti-coagulation with citrate have been developed. Citrate is an organic acid which is hepatically metabolized to bicarbonate. Research has show that patients differ in their sensitivity to bicarbonate in the dialysate. For instance, in some patients excessive bicarbonate may result in alkalemia, whereas, in some patients insufficient bicarbonate may result in acidemia. Therefore, if citrate is used as the anti-coagulent, then it is crucial that the concentration of bicarbonate in the dialysate be low or absent, depending on the sensitivity of the individual patient. Since the prior art dialysate solutions did not take into account the bicarbonate derived from citrate, the total effective bicarbonate concentrations tended to be too high resulting in metabolic complications. Recently, to address the aforementioned problems, pharmacists have begun to produce bicarbonate-free dialysate solutions in the laboratory. However, as discussed above, these methods are labour intensive, unregulated, non sterile, not pyrogen free, expensive and may lack sufficient quality control.

Accordingly, there exists a need for a sterile calcium bicarbonate-free concentrate for quickly and easily preparing dialysate solutions for use in dialysis and hemofiltration

### SUMMARY OF THE INVENTION

This invention generally relates to concentrates and corresponding diluted dialysis solutions with bicarbonate-free concentrations and methods and uses therefor.

CRRT requires the addition of an anti-coagulent such as citrate to the dialysate to prevent thrombosis. Citrate is an organic acid which is hepatically metabolized to bicarbonate. Research has shown that patients differ in their sensitivity to bicarbonate in the dialysate. For instance, in some patients excessive bicarbonate may result in alkalemia, whereas, in some patients insufficient bicarbonate may result in acidemia. Therefore, if citrate is used as the anti-coagulent, then it is crucial that the concentration of bicarbonate in the dialysate be absent or low, depending on the sensitivity of the individual patient. Usage of a bicarbonate-free dialysate solution takes into account the bicarbonate derived from citrate, and as a result the total effective bicarbonate concentration is accounted for and effectively controlled. Additionally, depending on the individual circumstances of the patient, the doctor can monitor the bicarbonate concentration and order infusions of more bicarbonate if the concentration is too low, or order infusions of acid if the concentration is too high. Thus, metabolic complications are effectively minimized.

In the current embodiment a concentrated solution is made such that adding a unit dose of this solution to a fixed volume of a phsyiologicially acceptable diluent produces a diluted sterile bicarbonate-free solution comprising Na 117±11 mmol/l, Mg 0.75±0.07 mmol/l, and Cl 118.5±11 mmol/l. In different embodiments, vials of different concentrations of dialysate solutions are available in the following unit dosages: 50 mL, 80 mL, 100 mL and 240 mL. In each case, when one unit dosage is added to 3L of a physiologically acceptable diluent it produces a diluted sterile bicarbonate-free solution comprising Na 117±11 mmol/l, Mg 0.75±0.07 mmol/l, and Cl 118.5±11 mmol/l.

In a first embodiment, the present invention provides a sterile calcium-free bicarbonate-free concentrate comprising sodium chloride (NaCl) 92.30±9.2 g/l, and magnesium chloride (MgCl2) 2.05±0.2 g/l. In this case, 240 mL of concentrate is added to 3L of a physiologically acceptable diluent to produce a diluted sterile bicarbonate-free solution comprising Na 117±11 mmol/l, Mg 0.75±0.07 mmol/l, and Cl 118.5±11 mmol/l. The concentrate can be stored at room temperature preferably for up to 48 months. The concentrate may also contain potassium, dextrose and/or b-hydroxy-butyrate or other ketones.

In a second alternative embodiment, the present invention provides a sterile calcium-free bicarbonate-free concentrate comprising sodium chloride (NaCl) 211.96±21 g/l, and magnesium chloride (MgCl2) 4.72±0.4 g/l. In this case, 100 mL of concentrate is added to 3L of a physiologically acceptable diluent to produce a diluted sterile bicarbonate-free solution comprising Na 117±11 mmol/l, Mg 0.75±0.07 mmol/l, and Cl 118.5±11 mmol/l. The concentrate can be stored at room temperature preferably for up to 48 months. The concentrate may also contain potassium, dextrose and/or b-hydroxy-butyrate or other ketones.

In a third alternative embodiment, the present invention provides a sterile calcium-free bicarbonate-free concentrate comprising sodium chloride (NaCl) 263.24±26 g/l, and magnesium chloride (MgCl2) 5.87±0.5 g/l. In this case, 80 mL of concentrate is added to 3L of a physiologically acceptable diluent to produce a diluted sterile bicarbonate-free solution comprising Na 117±11 mmol/l, Mg 0.75±0.07 mmol/l, and Cl 118.5±11 mmol/l. The concentrate can be stored at room temperature preferably for up to 48 months. The concentrate may also contain potassium, dextrose and/or b-hydroxy-butyrate or other ketones.

The inventors have determined that the concentrates and sterile solutions of the concentrates can be used in a number of novel applications including as a dialysate in hemodialysis of critically ill patients and as an infusate for hemofiltration. The concentrates offer a convenient means to prepare sterile and pyrogen free solutions at the bedside or in the pharmacy to avoid extemporaneous mixing. The bicarbonate-free concentrates of the first, second, third, and fourth alternative embodiments of the invention may be provided as a sterile concentrate in unit dosage to be added to a fixed volume of sterile water in PVC bags or as a prediluted sterile solution containing Na 117±11 mmol/l, Mg 0.75±0.07 mmol/l, and Cl 118.5±11 mmol/l.

### DETAILED DESCRIPTION OF THE INVENTION

This invention generally relates to concentrates and corresponding diluted dialysis solutions with bicarbonate-free concentrations.

CRRT requires the addition of an anti-coagulent such as citrate to the dialysate to prevent thrombosis. Citrate is an organic acid which is hepatically metabolized to bicarbonate. Research has shown that patients differ in their sensitivity to bicarbonate in the dialysate. For instance, in some patients excessive bicarbonate may result in alkalemia, whereas, in some patients insufficient bicarbonate may result in acidemia. Therefore, if citrate is used as the anti-coagulent, then it is crucial that the concentration of bicarbonate in the dialysate be absent or low, depending on the sensitivity of the individual patient. Usage of a bicarbonate-free dialysate solution takes into account the bicarbonate derived from citrate, and as a result the total effective bicarbonate concentration is accounted for and effectively controlled. Additionally, depending on the individual circumstances of the patient, the doctor can monitor the bicarbonate concentration and order infusions of more bicarbonate if it is too low, or order infusions of acid if it is too high. Thus, metabolic complications are effectively minimized. Vials of concentrated dialysate solutions are available in the following unit dosages: 80 mL, 100 mL and 240 mL. In each case, when one unit dosage is added to 3L of a physiologically acceptable diluent it produces a diluted sterile bicarbonate-free solution comprising Na 117±11 mmol/l, Mg 0.75±0.07 mmol/l, and Cl 118.5±11 mmol/l.

In a first embodiment, the sterile calcium-free bicarbonate-free concentrate consists essentially of sodium chloride (NaCl) 92.30±9.2 g/l, and magnesium chloride (MgCl2) 2.05±0.2 g/l. In this case, 240 mL of concentrate is added to 3L of a physiologically acceptable diluent to produce a diluted sterile bicarbonate-free solution comprising Na 117±11 mmol/l, Mg 0.75±0.07 mmol/l, and Cl 118.5±11 mmol/l. The concentrate can be stored at room temperature preferably for up to 48 months. The concentrate may also contain potassium, dextrose and/or b-hydroxy-butyrate or other ketones.

In a second alternative embodiment, the sterile calcium-free bicarbonate-free concentrate consists essentially of sodium chloride (NaCl) 211.96±21 g/l, and magnesium chloride (MgCl2) 4.72±0.4 g/l. In this case, 100 mL of concentrate is added to 3L of a physiologically acceptable diluent to produce a diluted sterile bicarbonate-free solution comprising Na 117±11 mmol/l, Mg 0.75±0.07 mmol/l, and Cl 118.5±11 mmol/l.

In a third alternative embodiment, the sterile calcium-free bicarbonate-free concentrate consists essentially of sodium chloride (NaCl) 263.24±26 g/l, and magnesium chloride (MgCl2) 5.87±0.5 g/l. In this case, 80 mL of concentrate is added to 3L of a physiologically acceptable diluent to produce a diluted sterile bicarbonate-free solution comprising Na 117±11 mmol/l, Mg 0.75±0.07 mmol/l, and Cl 118.5±11 mmol/l.

In one aspect of the invention, the concentrates may be used in continuous renal replacement therapies (CRRT) such as peritoneal dialysis and hemofiltration. The concentrates can be diluted in sterile physiologically acceptable diluents and used as a dialysis solution. The dialysis solutions of the invention provide a more physiological dialysis solution when compared to lactate dialysis solutions containing glucose and lactate and/or calcium. The bicarbonate-free concentrates of the present invention provide dialysis solutions that avoid the problems of prior art bicarbonate dialysis solutions in that it is highly stable i.e. calcium does not precipitate, and the concentrates can be stored for about up to 24 months. Additionally, bicarbonate-free concentrates take into account the bicarbonate which is derived from citrate, thus avoiding metabolic complications. Preferably the dialysis solutions are used for acute hemodialysis in intensive care patients.

The bicarbonate-free concentrates and dialysis solutions of the invention are cost effective because they facilitate process changes that increases efficiency by simplifying patient management, thus reducing nursing and medical staff time. They reduce or eliminate the need for corrective measures due to lactate or dextrose contained in other dialysates, lowering costs of extra syringes, needles, insulin, bicarbonate, etc. Citrate regional anticoagulation obviates the need for systemic or regional heparin or other anticoagulation and all of the complications associated with these methods. They also replace problematic lactate based peritoneal dialysis solutions used for dialysate in continuous hemodialysis all of which lead to a shorter number of days required in the intensive care unit (ICU).

It has been found that the bicarbonate-free concentrates of the present invention and dialysis solutions prepared from the concentrates are very suitable for CRRT, and in particular in CRRT adapted for acute renal replacement therapy of critically ill patients in particular, patients in intensive care units. The stability and sterility of the dialysis concentrate of the invention necessarily results in reduced renal replacement therapy costs.

The bicarbonate-free concentrates may be prepared by mixing the various components of the concentrates using conventional methods. The bicarbonate-free concentrates of the invention may be prepared according to the constituent ranges, or according to the preferred amounts set forth herein to prepare a unit dose i.e. a dose amount that can be mixed with a predetermined amount of a sterile physiologically acceptable diluent (e.g. 1, 3 or 5 liters of sterile water) to prepare a dialysis solution.

The bicarbonate-free concentrates may be used to produce dialysis solutions by mixing sterile physiologically acceptable diluents with the concentrates. Accordingly, in another aspect the invention provides dialysis solutions comprising the bicarbonate-free concentrates of the invention and a physiologically acceptable diluent. Physiologically acceptable diluents which may be used in the dialysis solution of the invention include sterile water and dextrose 5% in water (for injection).

The bicarbonate-free solutions are generally prepared by mixing a unit dosage of concentrate, with 3 liters of a sterile physiologically acceptable diluent. Further to the first, second and third alternative embodiments, the bicarbonate-free dialysis solution may be prepared pre-diluted and stored in CO2 impermeable bags. It consists of the following in mmol per liter: Na 117±11 mmol/l, Mg 0.75±0.07 mmol/l, and Cl 118.5±11 mmol/l. The dialysis solution may contain potassium, up to 4 mmol/liter, and/or b hydroxy-butyrate or other ketones, up to 5 mmol/liter. Preferably, the dialysis solution consists of the following in mmol per liter: Na 117, Mg 0.75, and Cl 118.5. If the dialysis solution is made in a PVC (polyvinyl chloride type) plastic container, it is advisable to use it within about 72 hours in order to avoid loss of bicarbonate through the plastic. The dialysis solution may be stored at room temperature or refrigerated. Calcium may be added to the diluent for CRRT, just prior to administration (M Leblanc et al, AJKD, 1995).

In yet a further aspect, the present invention provides a method for providing continuous renal replacement therapy to a patient comprising administering a sterile bicarbonate-free dialysis solution comprising Na 117±11 mmol/l, Mg 0.75±0.07 mmol/l, and Cl 118.5±11 to a patient in need thereof.

The present invention also provides a use of a bicarbonate-free concentrate according to the first embodiment comprising sodium chloride (NaCl) 92.30±9.2 g/l, and magnesium chloride (MgCl2) 2.05±0.2 g/l for preparing a dialysis solution for use in continuous renal replacement therapy. In this case, 240 mL of concentrate is added to 3L of a physiologically acceptable diluent to produce a diluted sterile bicarbonate-free solution comprising Na 117+11 mmol/l, Mg 0.75±0.07 mmol/l, and Cl 118.5±11 mmol/l.

The present invention also provides a use of a bicarbonate-free concentrate according to the second alternative embodiment comprising sodium chloride (NaCl) 211.96±21 g/l, and magnesium chloride (MgCl2) 4.7±0.4 g/l for preparing a dialysis solution for use in continuous renal replacement therapy. In this case, 100 mL of concentrate is added to 3L of a physiologically acceptable diluent to produce a diluted sterile bicarbonate-free solution comprising Na 117±11 mmol/l, Mg 0.75±0.07 mmol/l, and Cl 118.5±11 mmol/l.

The present invention also provides a use of a bicarbonate-free concentrate according to the third alternative embodiment comprising sodium chloride (NaCl) 263.24±26 g/l, and magnesium chloride (MgCl2) 5.87±0.5 g/l for preparing a dialysis solution for use in continuous renal replacement therapy. In this case, 80 mL of concentrate is added to 3L of a physiologically acceptable diluent to produce a diluted sterile bicarbonate-free solution comprising Na 117±11 mmol/l, Mg 0.75±0.07 mmol/l, and Cl 118.5±11 mmol/l.

The dialysis solution of the invention is preferably used to treat acute renal failure in critically ill patients. In contrast to prior art dialysis methods, the treatment typically does not involve incorporating calcium into the blood using the dialysis procedure. Therefore, the invention also contemplates a method for treating acute renal failure in a critically ill patient comprising dialyzing blood from the patient without introducing calcium into the blood removed from the patient during dialysis, and using a sterile dialysis solution prepared by mixing a sterile diluent with a sterile bicarbonate-free concentrate according to the first embodiment comprising NaCl 92.30±9.2 g/l, and MgCl2 2.05±0.2 g/l. The dialysis solution may additionally contain potassium, up to 4 mmol/liter, glucose up to 5 mmol/liter and/or b hydroxybutyrate or other ketones, up to 5 mmol/liter.

The invention further contemplates a method for treating acute renal failure in a critically ill patient comprising dialyzing blood from the patient without introducing calcium into the blood removed from the patient during dialysis, and using a sterile bicarbonate-free dialysis solution prepared by mixing a sterile diluent with a sterile bicarbonate-free concentrate according to the second alternative embodiment comprising NaCl 211±21 g/l, and MgCl 2 4.7±0.4 g/l. The dialysis solution may additionally contain potassium, up to 4 mmol/liter, glucose up to 5 mmol/liter and/or b hydroxy-butyrate or other ketones, up to 5 mmol/liter.

The invention further contemplates a method for treating acute renal failure in a critically ill patient comprising dialyzing blood from the patient without introducing calcium into the blood removed from the patient during dialysis, and using a sterile bicarbonate-free dialysis solution prepared by mixing a sterile diluent with a sterile bicarbonate-free concentrate according to the third alternative embodiment comprising NaCl 263.24±26 g/l, and MgCl2 5.87±0.5 g/l. The dialysis solution may additionally contain potassium, up to 4 mmol/liter, glucose up to 5 mmol/liter and/or b hydroxy-butyrate or other ketones, up to 5 mmol/liter.

The term “critically ill patient” or “critically ill patients” refers to patients that have a high mortality rate, acute renal failure, multiple organ failure, and multiple metabolic derangements. Critically ill patients which can be treated using the dialysis solution of the invention typically have acute renal failure and a high APACHE II score (Knaus W. A. Et al., Crit. Care Med. 13:818-827, 1985). An assessment of the number of failing organs may be performed using the procedure described in Jordan, D. A. Et al., Crit Care Med 15:897-904, 1987.

The concentrates and dialysis solutions of the invention are preferably administered to patients in intensive care who require dialysis and are hemodynamically unstable, or whose liver function is either impaired or at risk of impairment. Liver transplantation patients are especially difficult to manage and very often cannot handle any dialysate which contains lactate. Unable to transform the lactate in lactate buffered dialysis solutions to bicarbonate, they will go into acidosis if such solutions are used, and they require large doses of bicarbonate to correct pH imbalance.

The dialysis solutions of the invention are compatible with all systems used for CRRT including the commercially available systems such as the COBE Prisma Denver, Colo., Baxter CRRT System, Chicago, Ill., Hospital BSM22, Medolla, Italy, IMED Pump System, San Diego, Calif., Fresenius CRRT system, Dusseldorf, Germany or any other CRRT machine that uses peritoneal dialysate or other lactate-containing fluid or other bicarbonate based solutions as CRRT hemodialysate or infusate. When the dialysis solution is used with conventional systems for CRRT the consumption rate will typically be a unit dose of concentrate per hour assuming a dialysate flow of 1 liter per hour up to 2 liter per hour and a further 500 cc per hour of infusate.

The present invention includes kits for preparing dialysis solutions. In a first embodiment, the present invention provides a kit for preparing a dialysis solution comprising (a) one 240 ml unit of a concentrate comprising sodium chloride (NaCl) 92.30±9.2 g/l, and magnesium chloride (MgCl2) 2.05±0.2 g/l. and (b) three liters of sterile water.

In a second alternative embodiment, the present invention provides a kit for preparing a dialysis solution comprising sodium chloride (NaCl) 211.96±21 g/l, and magnesium chloride (MgCl2) 4.7±0.4 g/l (a) one 100 ml unit of a concentrate comprising and (b) three liters of sterile water.

In a third alternative embodiment, the present invention provides a kit for preparing a dialysis solution comprising sodium chloride (NaCl) 263.24±26 g/l, and magnesium chloride (MgCl2) 5.87±0.5 g/l (a) one 80 ml unit of a concentrate comprising and (b) three liters of sterile water.

The dialysis solution of the invention, either the concentrate or the diluted solution, may be contained in a plastic container (bag) for use at the bedside.

In one aspect of the invention, the bicarbonate-free solution will be prepared to a desired concentration for dialysis with citrate regional anticoagulation; Na 117±11 mmol/l , Mg 0.75±0.07 mmol/l, and Cl 118.5±11 mmol/l. The sterile water and all electrolytes, except calcium, are mixed, and if desired, diluted, and placed in a bag impermeable to carbon dioxide. At the time of dialysis, calcium may be added from, for example, a pre-filled syringe. Calcium may be added to produce a final calcium concentration according to local protocol. To produce a final calcium concentration in the range 1.25-1.75 mmol/L (5.0-7.0 mg/dl) calcium chloride 10% solution (100 mg/ml, 1.4 mEq/ml) 1.8-2.5 ml is added per 1080 ml of dialysate. Alternatively calcium gluconate 10% solution (100 mg/dl, 0.465 mEq/ml) 5.4-7.6 ml may be added per 1080 ml of dialysate to produce the same final calcium concentrations 1.25-1.75 mmol/L (5.0-7.0 mg/dL).

In a further aspect of the invention, the concentrates may be used as an infusate in hemodialysis. Consequently, the present invention provides a use of a sterile calcium-free concentrate according to the first embodiment for preparing an infusate for hemofiltration, wherein said concentrate comprises sodium chloride (NaCl) 92.30±9.2 g/l, and magnesium chloride (MgCl2) 2.05±0.2 g/l. The present invention also provides a method for hemofiltration comprising administering a sterile bicarbonate-free dialysis solution comprising Na 117±11 mmol/l , Mg 0.75±0.07 mmol/l, and Cl 118.5±11 mmol/l to a patient in need thereof. The infusate may be prepared by mixing 3000 ml of sterile water to 240 ml of the concentrate.

The present invention also provides a use of a sterile calcium-free bicarbonate-free concentrate according to the second alternative embodiment for preparing an infusate for hemofiltration with citrate regional anticoagulation, wherein said concentrate comprises sodium chloride (NaCl) 211.96±11 g/l, and magnesium chloride (MgCl2) 4.7±0.4 g/l. The present invention also provides a method for hemofiltration comprising administering a sterile bicarbonate-free dialysis solution comprising Na 117±11 mmol/l , Mg 0.75±0.07 mmol/l, and Cl 118.5±11 mmol/l to a patient in need thereof. The infusate may be prepared by mixing 3000 ml of sterile water to 100 ml of the concentrate.

The present invention also provides a use of a sterile calcium-free bicarbonate-free concentrate according to the third alternative embodiment for preparing an infusate for hemofiltration, wherein said concentrate comprises sodium chloride (NaCl) 263.24±26 g/l, and magnesium chloride (MgCl2) 5.87±0.5 g/l. The present invention also provides a method for hemofiltration comprising administering a sterile bicarbonate-free dialysis solution for citrate regional anti-coagulation comprising Na 117±11 mmol/l , Mg 0.75±0.07 mmol/l, and Cl 118.5±11 mmol/l to a patient in need thereof. The infusate may be prepared by mixing 3000 ml of sterile water to 80 ml of the concentrate.

The amounts and components of the concentrates and dialysis solutions of the invention may be modified to adapt to their use in cardiovascular surgery, peritoneal dialysis, hemodiafiltration, hemofiltration, and as an electrolyte solution.

We claim:

1. A sterile dialysis concentrate composition for use in a dialysis solution comprising sodium chloride (NaCl) 92.30±9.2 g/l, and magnesium chloride (MgCl2) 2.05±0.2 g/l.

2. A kit for preparing a dialysis solution comprising the sterile dialysis concentrate composition of claim 1 and optionally instructions for its use.

3. The kit of claim 2 further comprising sterile water sufficient to dilute the concentrate to a solution comprising Na 117±11 mmol/l, Mg 0.75±0.07 mmol/l, and Cl 118.5±11 mmol/l.

4. A method of preparing a sterile dialysis solution comprising diluting a sterile, dialysis concentrate composition of claims in a sufficient amount of sterile water to prepare a dialysis solution comprising Na 117±11 mmol/l, Mg 0.75±0.07 mmol/l, and Cl 118.5±11 mmol/l.

5. A method for providing continuous renal replacement therapy to a patient in need thereof comprising administering a sterile dialysis solution prepared according to the method of claim 4 to a patient in need thereof.

6. A method for treating acute renal failure in a critically ill patient without introducing calcium into the blood removed from the patient during dialysis comprising administering a sterile dialysis solution prepared according to the method of claim 4 to a patient in need thereof.

7. A method of preparing a sterile infusate comprising diluting a sterile, concentrate composition of claim 1 in a sufficient amount of water to prepare an infusate comprising Na 117±11 mmol/l, Mg 0.75±0.07 mmol/l, and Cl 118.5±11 mmol/l.

8. A method for providing hemofiltration to a patient comprising administering a sterile infusate prepared according to the method of claim 7 to a patient in need thereof.

9. A use of sterile calcium-free bicarbonate-free concentrate according to claim 1 for preparing an infusate for hemofiltration.

10. A use of a sterile, calcium-free bicarbonate-free concentrate according to claim 1 for preparing a dialysis solution for use in metabolic acidosis.

11. A sterile dialysis concentrate composition for use in a dialysis solution comprising sodium chloride (NaCl) 211.96±21 g/l, and magnesium chloride (MgCl2) 4.72±0.4 g/l.

12. A kit for preparing a dialysis solution comprising the sterile dialysis concentrate composition of claim 11 and optionally instructions for its use.

13. The kit of claim 12 further comprising sterile water sufficient to dilute the concentrate to a solution comprising Na 117±11 mmol/l, Mg 0.75±0.07 mmol/l, and Cl 118.5±11 mmol/l.

14. A method of preparing a sterile dialysis solution comprising diluting a sterile, dialysis concentrate composition of claim 11, in a sufficient amount of water to prepare a dialysis solution comprising Na 117±11 mmol/l, Mg 0.75±0.07 mmol/l, and Cl 118.5±11 mmol/l.

15. A method for providing continuous renal replacement therapy to a patient in need thereof comprising administering a sterile dialysis solution prepared according to the method of claim 14 to a patient in need thereof.

16. A method for treating acute renal failure in a critically ill patient without introducing calcium into the blood removed from the patient during dialysis comprising administering a sterile dialysis solution prepared according to the method of claim 14 to a patient in need thereof.

17. A method of preparing a sterile infusate comprising diluting a dialysis concentrate composition of claim 11 in a sufficient amount of water to prepare an infusate comprising Na 117±11 mmol/l, Mg 0.75±0.07 mmol/l, and Cl 118.5±11 mmol/l.

18. A method for providing hemofiltration to a patient comprising administering a sterile infusate prepared according to the method of claim 17 to a patient in need thereof.

19. A use of sterile calcium-free bicarbonate-free concentrate according to claim 11 for preparing an infusate for hemofiltration.

20. A use of a sterile, calcium-free bicarbonate-free concentrate according to claim 11 for preparing a dialysis solution for use in metabolic acidosis.
